(19) United States
(12) Patent Application Publication
(10) Pub. No.: US 2022/0162103 A1
(43) Pub. Date: May 26, 2022

(54) Environmental remediation, heat recovery, water purification, biomaterials

(71) Applicant: Michael David Lorton, Toledo, OH (US)

(72) Inventor: Michael David Lorton, Toledo, OH (US)

(73) Assignee: Algignis, Inc., Toledo, OH (US)

(21) Appl. No.: 17/102,390

(22) Filed: Nov. 23, 2020

(51) Int. Cl. C02F 9/00; C02F 3/32

(52) U.S. Cl. CPC C02F 9/00; C02F 3/32

(57) ABSTRACT

The inventive concepts herein allow several seemingly intractable environmental problems (toxic algae blooms, water pollution, petroleum-based plastics pollution, water contamination, premature retirement of zero-carbon nuclear power plants, etc.) to be solved economically and in a commercially practical way by integrating recovered heat, to grow or sustain organisms, that provide services (e.g. water purification, water pollution remediation, industrial recycling, contaminant degradation, load following) and/or products (e.g. biomaterials, hydrogen).

### RELATED APPLICATIONS AND INCORPORATION BY REFERENCE

Priority is claimed from U.S. Provisional Patent Application No. 62/900,435 filed on 13 Sep. 2019 by Michael D. Lorton, Toledo, Ohio.

### STATEMENT AS TO FEDERALLY SPONSORED RESEARCH

None.

### FIELDS OF INVENTION

Environmental remediation; water treatment, biomaterials, heat recovery (Search 435/135; 376/317; 210/739)

### SEQUENCE LISTING

Not currently applicable

### BACKGROUND

### Multiple Unsolved Problems

Introduction and Overview. This patent is the story of the evolution of solutions to several highly complex, seemingly intractable problems. All of the easy problems have been solved. Complex modern problems require complex iterative solutions across five domains: 1) Scientific/technical; 2) Business/financial; 3) Legal/regulatory; 4) Human/Social; and 5) Time. Justice William O. Douglas mistakenly believed that patentable innovation arose only from a “flash of genius”, essentially the creation of something from nothing. Patent lawyers know that patentable innovation arises from chaos, that is, looking at the explosion of human knowledge and figuring out how to assemble workable solutions from the almost infinite combinations and permutations of scientific principles across multiple disciplines. It is axiomatic in patent law that patents can be conferred for 1) new applications of combinations of known elements, and 2) solutions of previously unrecognized problems.

Patent professionals must have a scientific background, and for that reason often tend to focus almost exclusively on technical minutiae. Unfortunately, that focus has made the patent office the dusty graveyard of many brilliant technical solutions that were economically non-viable. Those un-commercialized patents do nothing to improve the lives of everyday Americans. The patent system was not created merely as a full-employment act for lawyers and patent professionals. Patent applications must always be evaluated in the context of the society in which they are presented in an effort to create scientifically-workable, economically-viable, regulatorily-allowable, socially-acceptable innovations whose time has come in an effort to improve the lives of working Americans who make up the heart and soul of this Nation.

Our governments are broke and broken, and we cannot depend on government to solve our problems. Exactly nothing has been done to reverse the burgeoning toxic algae blooms in Lake Erie. No-one is coming to save us; we have to save ourselves. The genius of our system is the yoking together of creativity, deep technical knowledge, self-interest, and entrepreneurial finance to solve daunting problems, while also creating wealth and prosperity. Solutions to small problems can be fully reduced to practice, but solutions of the complexity and scale needed to be commercially viable can only be presented prophetically, as here. Large scale solutions that involve nuclear power, solar energy, and water purification among other things cannot be financed without intellectual property protection or exclusive preliminary permits/licenses. Astonishingly, a federal court has taken the nonsensical position that one has no standing to exclusive preliminary permits for these technologies despite express Congressional authorization of such permits. Apparently, that leaves the patent system as the sole basis for protecting investors in those technologies.

Patent applications must address the critical real-world problems of the enablement and written description requirements in a world in which 1) nations ostensibly bound to honor American and Patent Cooperation Treaty intellectual property rights, nevertheless steal those technologies to the detriment of Americans; 2) foreign governments and militaries use patent disclosures to target critical American infrastructure; and 3) hackers (both sponsored and freelance) use patent disclosures to target critical American infrastructure for malevolent ends or financial gain. The U.S. Patent Act indirectly addresses those issues by 1) providing patent security protocols (e.g. Invention Secrecy Act); and 2) allowing patent examiners to consider additional information in possession of the inventor(s) (37 CFR 1.105). That undisclosed information may persuade examiners that thee patent claims are fully enabled, and that the inventors possessed the full scope of the invention at the time the patent application was filed. Our patent system is based on the admirable, but naïve notion that highly detailed disclosure is only beneficial. Unfortunately, that is only true among honorable men and women. Cold reality says otherwise.

The Problems: Toxic Algae. The inventor and his family live in Toledo, Ohio on the western edge of Lake Erie. Their drinking water is drawn from that lake. Since 1995 the lake has had progressively worsening toxic algae blooms in the summer months. In August of 2014 Toledo gained international notoriety when the City instructed its water customers not to drink, bathe in, or in any way consume the tap water for three days. The blue-green algae (cyanobacteria) produce hepatotoxins (e.g. microcystin) and neurotoxins (e.g. anatoxin and saxitoxin). Microcystin is a deadly liver toxin and a proven tumor promoter (cancer acceleration). Though less prevalent, neurotoxins cause long-term brain injury, especially in the very young and the very old.

Toxic algae blooms kill fish, birds, amphibians, reptiles, and mammals that live in or around Lake Erie. Toxic algae blooms cause a fetid thick green slime that has decimated commercial fishing, boating, tourism, swimming, and water recreation to the tune of billions of dollars. The United States Environmental Protection Agency (USEPA) imposed very expensive water purification requirements on Toledo in an effort to reduce toxin levels in the drinking water. To date Toledo has expended more than $400 million in upgrades to the Collins Park Water Treatment Plant, but technologies directed to reduction in microcystin (e.g. ozone treatment) are still not complete.

Toledo reassures its citizens that the water is just fine, but they never get actual numerical levels of microcystin, nor are they given any of the methodology used to measure those levels. The World Health Organization (WHO) and drinking water experts have repeatedly stated that drinking water suppliers should report total microcystin levels publicly (i.e. both intracellular and extracellular microcystin). International authorities have established one microgram/liter of microcystin as the danger level for that toxin. WHO reports international instances of microcystin levels in source waters up to 120,000 micrograms/liter with many measurements in the 20,000-30,000 microgram/L range. The inventor found spreadsheets from the National Oceanographic and Atmospheric Administration (NOAA) indicating reported extracellular microcystin levels in Lake Erie of 1-2 micrograms/L, but unreported intracellular extracted microcystin levels of over 300 micrograms/L. That means that unreported total microcystin levels are approximately 300 times greater than the reported extracellular levels.

The city, state, and federal governments have done exactly nothing to reduce the toxic algae blooms in Lake Erie for more than 20 years. The primary causes of those toxic algae blooms are 1) the shallow waters of the Western Lake Erie basin; 2) rising global mean temperatures; and 3) agricultural runoff of excess phosphorus, nitrogen, and organic carbon (P/N/C) in fertilizers. Cheap, plentiful food is a prized political objective, and agricultural runoff of P/N/C is expressly exempted from regulation under the Clean Water Act (CWA) as non-point source pollutants.

Despite express warnings from the WHO, the U.S. Center of Disease Control and Prevention (CDC), and the Ohio Departments of Natural Resources (ODNR) and Environmental Protection (OEPA), the state and local governments proposed building an artificial wetland at the mouth of the Maumee River where it enters Lake Erie. It was pointed out that such an artificial wetland 1) would expose dense adjacent human populations to mosquitos with a significant risk of transmitting West Nile, Eastern Equine Encephalitis, and other viral diseases; 2) mosquito and invasive species control would require spraying highly toxic pesticides and herbicides directly into the City's drinking water source; 3) the wetland would only take up P/N/C when the plants were growing in the warm summer water, and that almost no P/N/C would be remove when the cold spring floods poured the most P/N/C into the lake; 4) eighty percent (80%) of Lake Erie's water and approximately 50% of the P/N/C flows in through the Detroit River. An artificial wetland in the Maumee River would have no impact on those pollutants or the overall contamination of the lake; 5) for the wetland to be effective in removing P/N/C, the biomass and density of the plants would have to increase immensely. That, in turn, would restrict flow in the River and cause silting out in the shipping channels; and 6) intentionally exposing people to mosquito-borne diseases and pesticides/herbicides in their drinking water would expose Toledo, Lucas County, individual state officers, and potentially the State of Ohio itself to massive contingent liability.

In spite of the express exemption of agricultural P/N/C to regulation under the CWA, environmental groups have sued in federal district court to impose CWA's Total Maximum Daily Load limits (TMDL) on Ohio's farmers and concentrated animal feeding organizations (CAFO's). U.S. farm bankruptcies are already at a high level. Imposing costly TMDLs on farmers (impermissible) and CAFOs (permissible) would impose a severe financial stress and cause farms to fail and the prices of food to rise. Given the massive amount of P/N/C that flows through the Detroit River, a TMDL injunction covering anything less than Wisconsin, Illinois, Indiana, Michigan, and Ohio would be ineffective at substantially reducing toxic algae blooms in Lake Erie. Ontario, Canada contributes significant P/N/C to Lake Erie and a U.S. court has no jurisdiction over the Canadians.

The Problems: Perfluoroalkyls (PFAS), Anti-Retroviral Drugs, and other Chemicals in the Water. Our drinking water in the United States is contaminated by a multitude of toxic chemicals in addition to algal toxins. 1) Poly- and Perfluoroalkyl substances are chemicals used as fire retardants. Unfortunately, the same properties that make them heat-resistant (extremely high bond dissociation energies between the carbon and halogen molecules) make them “forever chemicals” (i.e. they do not break down in the environment). PFAS chemicals are a growing concern as they are linked to human cancers, and several adverse health effects. PFAS chemicals have some the strongest covalent bonds known in chemistry (carbon-halogen bonds) with extremely large dissociation energies. That means it takes a massive amount of expensive energy to break down dangerous PFAS chemicals into harmless constituent molecules. PFAS chemicals can be separated from water and collected at relatively low cost; the problems are the immense cost of the energy needed to break those chemicals down

2) One of the largest Hazardous Waste Landfills in the nation (Envirosafe) is located on the shore of Lake Erie. Massive volumes of highly toxic known carcinogens (e.g. benzene, toluene) have been dumped there.

One unrecognized problem is the human excretion of active metabolites of anti-retroviral drugs into our drinking water sources. Anti-viral drugs used to treat Human Immunodeficiency Virus (HIV, an RNA virus) are given in combinations to prevent/slow resistance mutations in the AIDS virus. Unfortunately, many of them are only mildly changed by the body's metabolism and are excreted as active chemicals with continuing anti-RNA virus activity. The problem is that such drugs cause mutations and resistance in RNA viruses in the environment. As of early September 2020, we are in the midst of the COVID-19 pandemic. SARS CoV-2 is an RNA virus that has mutated to a form previously unencountered by humans. SARS, MERS, and COVID are all recent emerging RNA viruses that have mutated to cause severe disease in humans. WE ARE NOT CLAIMING THAT DRUGS USED TO TREAT HIV HAVE IN ANYWAY CONTRIBUTED TO SARS, MERS, OR COVID. What we are saying is that RNA viruses have no proof-reading function and have remarkably high mutation rates. We know anti-retroviral drugs cause mutations and resistance in patients treated with those regimens. We respectfully submit that active metabolites of those same anti-RNA drugs are highly likely to accelerate mutations in RNA viruses found in animals and the environment. We are acutely aware that no-one wants to consider this real probability given the social backlash that might occur against people taking anti-retroviral drugs. That is why we only broach the subject at the same time we propose a solution. We strongly condemn anyone from discriminating against anyone taking those drugs in any way. We believe an innovation that breaks down those drugs in a cost-effective way helps both those patients and society at large.

The Problems: Petroleum-based Plastics Pollution. Petroleum-based plastics are lightweight, durable, and easily molded into a variety of products. Unfortunately, they degrade in the environment only very slowly—potentially lasting several hundred years. Plastic waste jams our landfills, rivers, lakes, and oceans. Animals are injured or die after becoming entangled in plastics. Birds and fish eat large chunks of plastic. Microplastics are increasing shown to be harmful to all the creatures that directly and indirectly ingest them in the food web. Only a small percentage of plastic waste can be profitably recycled.

The Problems: Premature Retirement of Zero-Carbon Nuclear Power Plants. The current Pressurized Light Water Nuclear Reactors (PWR) and Boiling Light Water Nuclear Reactors (BWR) convert only about 35% of their energy into electricity; the other 65% of the heat is vented into the environment as thermal pollution. The United States is one of the few nuclear countries in the world that does not recover and utilize that “waste” heat to produce valuable products and services. Naturally occurring Uranium is 0.7% fissile U235 and 99.3% U238. Fuel rods for light water reactors in the U.S. are only enriched to approximately 5% U235 with 95% U238. Fuel rods must be rotated and eventually replaced every 18-24 months when the U235 has fissioned into highly radioactive (relatively short-lived) fission products. Spent fuel rods contain approximately 95% fertile U238 (relatively long-lived) that could be converted to fissionable Plutonium239. Without reprocessing or reuse of the “spent” fuel, only 5% of the available energy of the Uranium is currently utilized. Short-lived fission products need only be stored for approximately 300 years (ten 30-year half-lives) before the radioactivity has fallen to background levels. Long-lived radionuclides (e.g. U238) must be stored for nearly one million years (ten 100,000-year half-lives) before its radioactivity falls to background levels.

Current nuclear power plants cannot compete with electricity produced by cheap fracked (hydraulic fracturing) natural gas and cannot compete in “next day” auctions for projected baseload electricity production. Supply and demand in the electrical grid must be carefully matched. Too little electricity and we experience brown-outs or black-outs; too much electricity and we burn up motors, sensors, and lights. Currently, nuclear power cannot be rapidly adjusted from moment to moment so as to “follow the load” required by the grid. Given the absence of nuclear load following, the nuclear power plants (NPP) cannot bid for “peaker” auctions for very short-term electricity production. Until the Federal Energy Regulatory Commission (FERC) issued its Minimum Offered Price Rule (MOPR) in December 2019, the only auctions into which NPPs could bid was the long-term (2 year) capacity markets. Now FERC has taken the position that any forms of direct or indirect state subsidies for nuclear power cannot allow NPPs to bid below a certain price in capacity auctions. That will have dire consequences for nuclear power if not overturned on appeal.

Fracking has made the United States a net exporter of oil and natural gas. Energy independence and massive job creation are justifiably extremely politically and economically popular. Unfortunately, even those intellectually honest people who recognize the value of fracking, understand that fracking imposes many very inimical externalities on society. Millions of gallons of highly toxic and carcinogenic hydrocarbon solvents are pumped into the fracked gas well to increase yield. Much of that volume of toxic hydrocarbons is not recovered and threatens the groundwater. USEPA has been stripped of authority to regulate fracking contamination of groundwater. Fracking vents significant amounts of methane (CH4) a greenhouse gas that is 25 times more potent than CO2. Natural gas is cleaner than coal, but still produces significant amounts of CO2 when burned to produce electricity. The drop in oil and gas demand during the COVID-19 pandemic has caused many fracking companies to declare bankruptcy. Many states did not require the fracking companies to set aside adequate reserves to close the now abandoned well. Nationwide abandoned well closing costs to be borne by taxpayers amounts to billions of dollars. Finally, both the Wall Street Journal and the New York Times report that 1) fracking companies have consistently suffered losses; 2) reported oil and gas reserves are vastly overstated; and 3) the future costs of fracking are significantly under-estimated.

The Problems: Oil Refineries Threatened by the Ethanol Mandate and Loss of Oil Supply Pipeline. Northwest Ohio and Southeast Michigan have 1) the Toledo Refinery; 2) the Husky Refinery; 3) the BP (British Petroleum) Refinery; and 4) the Marathon Refinery, all of which are threatened with closure by the high prices of ethanol credits (they lack the ability to blend ethanol in their gasoline and transport it economically by pipelines) they are forced to buy or the permanent closure of Pipeline #5 that comes down through the Straits of Mackinac (the State of Michigan is trying to permanently close the pipeline for fear that it will rupture into Lake Huron). Closure of those refineries will cost thousands of good jobs and millions in taxes.

The Problems: The Perverse Economics of Solar Energy. Northwest Ohio is home to the only solar panel manufacturing plant in the United States (FirstSolar). Unfortunately, solar energy has several problems: 1) Solar energy is very diffuse and utility-sized solar installations require a massive amount of land; 2) Solar energy is intermittent. Obviously, there is no solar energy at night, and it is significantly diminished on cloudy days. We have no economical storage capabilities for solar (or wind) energy, and, in spite of hopes, we seemed constrained by the laws of physics for battery technology; 3) Solar energy supply is ill-timed to demand. It starts to rise at 8-9 am and begins to drop in the late afternoon—right when demand for electricity starts to rise (people return home and begin cooking, lighting, and heating/air conditioning their homes). 4) The unpredictability of solar energy makes it exceedingly difficult to match electricity supply and demand. To provide adequate supply to the grid on cloudy days, we are forced to install massive solar facilities. On sunny days, far more solar energy is produced at midday than the grid demands. Curtailment, the practice of cutting off the flow of solar energy from the panels to the grid, causes the semiconductors in the panels to over-heat. That over-heating permanently damages the semiconductors and degrades their efficiency. In an effort to avoid the adverse effects of curtailment, large solar installations end up paying customers far afield to take their excess power; 5) You cannot run a business in which you have to pay your customers to take your product (here, solar power). The graph of electricity price on the “Y” axis and time of day on the “X” axis produces the “Duck Curve”, that is, the price of electricity starts to drop into the negative range at midday, only to rise again as evening draws near. Nuclear power nor any other source of energy can compete for baseload when one of the suppliers (solar) is causing the price of the energy to go negative; and 6) Solar energy is heavily subsidized. The cost of solar panels and equipment has dropped significantly, but solar is still not able to compete without those subsidies.

The Problems: Expensive Hydrogen Production. Hydrogen may well be the clean fuel of the future, but it is uneconomical in the current market with the current technologies. The International Atomic Energy Agency (IAEA) has created an excellent software program, Hydrogen Economic Evaluation Programme (HEEP), that takes into account a large number of variables to produce a projection of the economic viability of any given hydrogen generation program. Hydrogen is most economically produced by the breakdown of water (hydrolysis). The hydrogen-oxygen bonds in water are very strong and require immense amounts of energy to break. The HEEP program indicates that the relatively low outlet temperatures of current nuclear power plants (NPP) are too low (approximately 200 C to 300 C) to economically produce hydrogen.

In summary, there are a large number of daunting societal problems that have, to date, defied scalable, commercially viable solutions.

### SUMMARY OF INVENTIVE CONCEPTS

Admittedly, this “summary” is not like those seen in most patent applications, but we believe it is as important to examine “how” the inventive concepts were created as it is to learn what they entail. We would ask for a little leeway because workable solutions to complex, modern-day problems require long, arduous iterations across multiple disciplines. Only with unacceptable hindsight bias was any of it obvious to try.

35 U.S.C. 103 states, “ . . . Patentability shall not be negated by the manner in which the invention was made.” Every inventor starts with a problem, then she works through multiple iterations to achieve a workable solution (The problem of ______ is solved by ______). Most inventors are technical geeks (no offense) and have little or no understanding of either patent law or commercialization of their inventions. They focus almost exclusively on the technical challenges and leave it to their patent lawyer and businesspeople to hammer the invention into a protectable, commercially viable product or service after the fact. When patentability and commercial viability are after thoughts, valuable technologies are often less than fully developed.

We would respectfully submit that the better way to craft workable solutions to complex problems is to weave considerations of patentability and commercialization into every technical step. This inventor is tech geek who is also a patent lawyer with an MBA. He started with the problem of deadly toxins in the drinking water of his community. First, he looked for the scientific solutions to his threshold problem; then, understanding what would be required to protect and commercialize those solutions; he reviewed the prior art, considered the regulatory implications, and do a business analysis of the potential solutions. Every analysis of that kind solved one problem but generated another problem. The inventor then attacked the new problem by repeating the process. This he did over and over again in an effort to iteratively develop scientifically valid, regulatorily-allowable, commercially viable solutions.

The inventor first had to consider why no viable solutions had emerged in an effort to learn from the mistakes of others. Everyone trying to solve the toxic algae problem saw algae and the phosphorus, nitrogen, and organic carbon (P/N/C) in agricultural runoff as the “enemy”. They all seemed intent on trying to get government to solve the problem by imposing massive costs on municipal wastewater systems and farmers. The first insight was that “one man's trash is another man's treasure.” Yes, toxic cyanobacteria in the open waters of Lake Erie are the enemy, but non-toxic algae that slurp up massive amounts of P/N/C could be our ally. The inventor did a prolonged, deep dive into the water purification biology of algae (and several other organisms) and found that 1) algae (and other organisms) grow four times as fast as terrestrial plants; 2) algae (and other organisms) consume massive amounts of P/N/C in their rapid growth and multiplication; 3) algae (and other organisms) have been used to clean municipal and industrial wastewater on small scales; and 4) non-toxic algae (and other organisms) have commercial value.

The inventor then calculated 1) the volume of water in the Western Basin of Lake Erie; 2) the concentration/mass of P/N/C that would have to be removed to reduce the total P/N/C by 40%; 3) the biomass of algae (and other organisms) that would need to be produced to effect the 40% reduction in P/N/C; and 4) the size, cost, and operating parameters of algae (or other organism) cultivation facilities that would be necessary to grow the required algal (organismal) biomass. As previously noted, one solution brings other problems. The water purification biology and engineering requirements were proven and validated, but we would be left with a gargantuan amount of low value algae that made the solution economically non-viable. The new problem was: How then to increase the value of the algae or other organisms use to clear the P/N/C?

The inventor has a background in biotechnology, and the insight that solved the low-value algae problem was genetic engineering or selective breeding of organisms. Zinc-finger nucleases, TALENS, and CRISPR genetic engineering have been used to create organisms that produce substances not usually produced by the organisms. The inventor did an extensive review of the genetic engineering technology and higher value substances that could be produced at facilities located near a body of water. One of many possible valuable products were bioplastics. Bioplastics only account for an estimated 2-5% of the world plastics market. The inventor did an extensive review of material science, biomaterials, polymer chemistry, plastics/bioplastics characteristics, and plastics/bioplastics end uses. The business and financial analysis then revealed potentially viable business models if costs, yields, and market share targets could be attained. Unfortunately, selective breeding or genetic engineering of organism (including algae) increased costs of development, engineering, production, and regulatory approval. The projects would only be commercially viable if production were nearly year-round. No large cultivation projects for algae or other organisms had ever been sited in an area like the Great Lakes region. The northern climes had things algae and other organisms detest: the cold and dark of winter. We could increase the value of the algae and their byproducts, but a new problem then arose: How to grow algae or other organism in colder climes year-round at commercial scale?

Again the inventor's background in chemistry and molecular biology provided the answer. It is a little-known fact that not all algae need sunlight to grow (phototrophic/autotrophic algae or other organisms). There are naturally occurring and genetically engineered algae/organisms that can grow with little or no light as long as they are provided heat and organic carbon (heterotrophic algae/organisms). Algae/organism which can grow both with the use of light and without light are called mixotrophic. Having extensively reviewed algal/organismal carbon partitioning and metabolism, the inventor did a business/financial analysis of heterotrophic/mixotrophic algae/organisms. Having ready access to cheap/free water and nutrients (e.g. P/N/C) reduced the production costs far below what other algae/organism cultivators had faced, but the cost of heating the cultivation ponds/vessels all winter again made the business model both financially and environmentally unfavorable. The inventor had solved the problem of growing modified algae/organisms where it is cold and dark much of the year, but the new problem was largely one of economics. Providing enough heat for the heterotrophic algae/organisms to get them through the cold falls, winters, and springs would be frightfully expensive. The new problem was then: How to heat the cultivation facilities so as to grow modified algae/organisms producing valuable products/services year-round at commercial scale (so as not to leave the massive investment in the physical plant idle for much of the year)?

Then it hit the inventor, Dr. Lorton (I apologize for referring to myself in the third person, but this is a legal document, and it reads better this way). The news that the Davis-Besse nuclear power plant (NPP), and most others NPPs in the United States would be closed because it/they could not compete with electricity produced by cheap fracked natural gas, triggered a memory that approximately ⅔ of the energy of NPPs is wasted as thermal pollution. Again, one man's trash is another man's treasure. All of the waste heat from the Davis-Besse and Fermi Nuclear Power Plants could be recovered by heat exchangers installed on their primary or secondary coolant loops. That recovered heat could be used to heat the heterotrophic algae/organism cultivation ponds year-round at a commercial scale that could substantially reduce P/N/C in Lake Erie and produce valuable byproducts and services. It all came together: 1) Waste heat recovered from the NPPs would allow 2) year-round cultivation of algae/organism at commercial scale that would 3) substantially reduce P/N/C in Lake Erie thereby substantially reducing toxic algae blooms, and 4) produce valuable byproducts (e.g. bioplastic polymers) or services (e.g. cleaning the lake water of other chemicals). We would be killing three birds with one stone: 1) Being deprived of excess P/N/C, the toxic algae blooms would be substantially reduced; 2) Bioplastics could be produced economically at commercial scale so as to help reduce petroleum-based plastics pollution; and 3) Economically non-competitive NPPs could monetize their waste heat and avoid premature retirement. The inventor did a deep dive into heat exchangers, thermodynamics, and nuclear power plants to confirm and make operative the solutions.

But that was not the end. Business schools teach that startups must always be looking at product/service line extension and further innovation. Knowing that, the inventor sat back and thought about other problems his technical solutions might profitably solve. Did not society need a commercially viable solution to water pollution other than with algal toxins? American waters are contaminated by Polyfluoroalkyl chemical, pharmaceuticals, and other hazardous chemicals. Nuclear power and renewable energy sources (e.g. solar and wind) had not been integrated in a way that would allow them to be synergistic rather than antagonistic. If the heat value of combined nuclear and renewable energy sources were adequately increased, could we not economically produce hydrogen and degrade a myriad of water polluting drugs and chemicals? Oil and gas refineries were on the verge of insolvency because of the high cost of buying mandated ethanol credits. Could the refineries not also monetize their recovered heat to clean water and produce valuable byproducts year-round at commercial scale? All of those complex problems presented scalable opportunities for additional businesses and income streams. Dr. Lorton set to work doing the extensive scientific, business/financial, and legal/regulatory analyses for each.

This Summary of the Inventive Concepts is quite different from the disclosures the examiners at USPTO usually deal with because the manner in which these inventive concepts were created is vastly different from the usual manner of invention. The inventor, Dr. Michael Lorton, has an unusual background. He has an undergraduate major in chemistry, with minor in molecular biology (biology, genetics, physiology, etc.). He had a year of university physics, and math through calculus, analytical geometry, differential equations, and statistics and probability. He is a medical doctor board-certified in internal medicine and cardiology. He is a patent lawyer with practice as a litigator. He has a Master of Business Administration with a Finance concentration (including Research Commercialization). We say this not to be immodest, but to give credibility to the notion that one man could iteratively work through the five domains of modern problem solving to craft workable solutions (across several scientific disciplines) to technical, financing, and legal/regulatory problems, some of which were previously unrecognized.

To summarize the process, at each juncture, Dr. Lorton would think through the possible solutions (like a physician's differential diagnosis), then find scientific/technical solutions. Next, he would make a business analysis of each technical solution, because a technical solution that can never get financed is essentially worthless. Dr. Lorton would next do a regulatory and intellectual property legal analysis. An inventor can have great science and a great business model, and still fail because the bureaucracy will not approve the solution or patent protection cannot be assured. Part of that legal/regulatory business analysis was doing patent and non-patent searches at each at each and every juncture and for each and every technical solution. It is exceedingly difficult to get entrepreneurial financing from private equity and venture capital investors unless the technology is “protectable”. Nobody wants to be the first mover to create a new market only to have competitors swarm in and steal market share once all the hard work is done.

The Summary discussion up to this point is aimed at increasing the importance of the Information Disclosure Statement (IDS) and the interview process (in which the inventor personally shows the examiner the highly detailed technical, business, and legal analyses) that show he, the inventor, had the inventive concepts firmly and clearly in mind, and that the inventive concepts are enabled to the full scope of the claims. Currently, the IDS is largely a check-the-box exercise for busy USPTO examiners. When, as here, the inventor wove the limitations of prior art into his solutions from the very beginning, the IDS plays a more prominent role. Here, Dr. Lorton has annotated the IDS references to give a better, fuller understanding of how Dr. Lorton's inventive concepts walk up to the edge of the prior art but do not cross over. That is why Dr. Lorton is incorporating by reference the IDS patents and patent applications for “critical/essential” disclosures and non-patent literature for supporting disclosures.

We mean no disrespect, but there are problems with the patent system itself that must be addressed. In an ideal world broad disclosure would spread knowledge and aid mankind in the upward march of progress. Unfortunately, we do not live in an ideal world. If one (but by no means the only or main) objective of these inventive concepts is to create a nuclear waste heat recovery industry in the United States, then one must realize that detailed disclosures beyond what is necessary for patentability aid very bad actors around the world. There are nation states that flout intellectual property protections and happily play us for fools. There are bad actors ready and willing to cause havoc when they learn details of nuclear power integration. To avoid these patent system problems, Dr. Lorton, wants to work closely with USPTO to demonstrate only enough to secure patent protection without giving aid and comfort to America's enemies. Only by working together closely can we avoid a secrecy order.

This is why Dr. Lorton has created highly detailed technical/business/legal documents including, but not limited to, algal/organism biology, genetic engineering, selective breeding, algal toxicology, polyfluoroalkyl toxicology, anti-retroviral resistance promotion, hazardous waste toxicology, algae cultivation/energy co-generation facility engineering and operations, uses for algae/organism biomass, regulatory approval under the Toxic Substances Control Act (TSCA) and Clean Water Act (CWA), polymer chemistry, bioplastics/biomaterials production, biomaterials/bioplastic degradation/recycling, hydrogen production, heat exchanger engineering, thermodynamics, nuclear reactor physics, nuclear reactor types and engineering, nuclear reactor safety, nuclear cooling systems engineering, nuclear waste heat recovery systems, waste heat recovery from refineries and other heat-intensive industries, regulatory approval under the Atomic Energy Act (AEC), solar energy/semiconductor material science, solar energy engineering, integration of nuclear and solar energy into the electrical grid, regulatory approval under the Federal Power Act (FPA), sensor and automated control technology for nuclear and solar energy processes, and cybersecurity issues. Putting all of those into a patent would take about a bazillion pages, waive legal privilege, and give unnecessary detail to bad actors.

Given our need to interview and interact with the USPTO person-to-person, it is also why the inventor is asking the Director for a 3-4-month suspension of the examination and prosecution of this patent application. The COVID-19 pandemic prevents the inventor and examiners from meeting in person in a way necessary to deal with some very important security and other issues. If the truth be known, the inventor would rather have the examination and prosecution of this application accelerated so issue would occur sooner for this critical environmental remediation patent. Unfortunately, the pandemic precludes that.

The following is the more typical “Summary of Inventive Concepts” in reverse solution-solves-problems format:
The use of algae or other fast-growing organisms to clean water solves the problems of:
   Toxic algae are deprived of excess phosphorus, nitrogen, and carbon (P/N/C) that promote their growth and toxin production,
   Municipal water treatment plants face lower capital and operating costs of treating algal toxins,
   The citizens who drink the water are exposed to much less hepatotoxins, neurotoxins, and tumor/cancer promoters,
   The environmental damage of algal toxins on fish and wildlife,
   The loss of boating, commercial/recreational fishing, and tourism/recreation,
   Farmers and owners of animal feeding operations are under less pressure to reduce manure/fertilizer usage. Modern genetically modified crops grow very rapidly and need intense nutrient concentrations. Plant borders around fields used to soak up the P/N/C seed the crops with more weeds which require more herbicides and drive up costs.
   Federal judges are spared the need to impose massive Total Maximum Daily Load limits (TMDL) on multiple states around the Great Lakes.
The modification of algae or other organisms to produce high value products or services solves the problems of:
   Massive amounts of low value algae/organisms,
   Creates new industries for bioplastics or other biomaterials around the industrialized upper Midwest,
   Potentially solves the petroleum-based plastics pollution problems,
   Builds a new biotechnology industry (applications of genetic engineering) in northern Ohio and southeast Michigan.
The recovery of waste heat from nuclear power plants to enhance year-round growth and maintenance of algae or other organisms solves the problems of:
   Prevents the premature retirement of zero-carbon nuclear power plants (NPP) that would contribute to global climate change,
   Allows NPPs to monetize their waste heat,
   Gives the patent holder standing to secure preliminary permits and licenses under the Federal Power Act. Those federal preliminary permits and licenses then allow the permittees/licensees to partner with public or private operators of nuclear power plants and to evade the adverse effects of the Maximum Offered Price Rule (MOPR) issued by the Federal Energy Regulatory Commission (FERC),
   Allows the waste heat to be used for degradation of toxic chemicals (PFAS, drugs, hazardous waste)
   Allows for industrial plastics degradation and recycling,
   Allows nuclear power plants to bid in “next day” auctions for power supply because they can now adjust their outputs to follow grid electricity load requirements more closely,
   Preserves all the advantages of current nuclear power plant sites (transmission infrastructure, cooling water, safety location, human expertise, pre-existing regulatory approvals, social acceptance by locals, and access to nuclear waste as fuel for the advanced reactors) for the construction of advanced Generation 4 NPPs,
   Allows the advanced Generation 4 NPPs to burn as fuel the nuclear “waste” now stored on-site.
The recovery of waste heat from refineries to enhance year-round growth of algae or other organisms solves the problems of:
   Refineries closing because of the ethanol blending mandates,
   Refineries closing because of loss of the Enbridge #5 Pipeline,
   Refineries repurposing to biorefineries,
   Allowing oil and gas companies to transition to biomaterials.
The recovery of waste heat from the combination of nuclear power plants and solar energy installation solves the problems of:
   Produces massive amounts of low-priced energy that can be used for PFAS/drug/hazardous chemical degradation, hydrogen production, and biomaterials production, industrial plastics degradation, etc.,
   Prevents curtailment damage to solar panels,
   Allows solar and wind to compete without subsidies,
   Makes liquid metal batteries economically possible.
The production of bioplastics by the modified algae or other organisms solves the problems of:
   Petroleum-based plastics pollution.
The industrial degradation of plastics solves the problems of:
   Environmental damage of macro-plastics,
   Food web/living creature damage of micro-plastics.
The collection and degradation of PFAS chemicals solves the problems of:
   Cancer and other adverse health effects to humans.
The collection and degradation of drugs and pharmaceuticals solves the problems of:
   Adverse health effects of waste drugs on human health,
   Reduces mutational pressure on RNA viruses from anti-retroviral drugs in the environment.
The collection and degradation of other hazardous waste/chemicals solves the problems of:
   Adverse health effects of hazardous chemicals leaking into drinking water sources and bioaccumulating.
The production of hydrogen solves the problems of:
   Reduces CO2 and CH4 greenhouse gas emissions when clean H2 is used as fuel.

### DETAILED DESCRIPTION OF THE INVENTIVE CONCEPTS

A patent applicant is required to submit a detailed Information Disclosure Statement (IDS) disclosing patent and non-patent literature that is material to patentability such as novelty, usefulness, non-obviousness, enablement, written description, best mode, definiteness, etc. As such, the IDS is an essential part of the patent specification. An applicant cannot submit an IDS with a provisional application. I will submit a detailed material Information Disclosure Statement that gives material support for all the elements of patentability and validity and that Information Disclosure Statement is hereby incorporated by reference in support of both essential and non-essential elements in this utility patent application and any and all divisional applications, continuation applications, continuation-in-part applications, re-issue applications, and/or other applications permitted under the patent laws and regulations.

Recovery of unused heat and combined heat and power production has been used in limited circumstances. Macro- and micro-organisms have been modified by selective breeding as well as genetic and epigenetic processes. Such organisms have been used to produce substances that the organisms usually produce as well as substances that the organisms do not usually produce. Organisms have been used to substantially remove or lessen the concentration of substances in water. What has not been done before is combining technologies to recover unused heat or heat from combined heat and power production to grow organisms that substantially remove or lessen the concentration of substances in water and also produce valuable substances or services in circumstances in which those organisms could not be otherwise sustained.

The recovery and use of previously unused heat can also be put to very valuable uses even if no organisms are used to clean water or produce bioproducts. With respect to this patent application, the following illustrative terms include, but are not limited to:

Heat sources include: 1) nuclear reactors; 2) solar energy panels; 3) wind energy turbines; 4) oil and/or gas refineries; 5) metal melting and shaping operations; 6) cement production; 7) refractory production; 8) glass production; 9) coal burning operations; 10) natural gas burning operations; 11) chemicals production; 12) frictional energy; 13) combustion energy; 14) solar energy concentrating systems; 15) magnetic energy; 16) gravitational energy; 17) kinetic energy; 18) geothermal energy; and 19) chemical energy.

Nuclear reactor types include: 1) Generation 1; 2) Generation 2; 3) Generation 3; 4) Generation 4; 5) Light water reactors; 6) Pressurized water reactors; 7) Boiling water reactors; 8) Heavy water reactors; 9) Burner reactors; 10) Fast neutron reactors; 11) Molten metal reactors; 12) Advance gas-cooled reactors; 13 Small modular reactors; 14) Molten salt reactors; 15) Floating reactors; and 16) Breeder reactors.

Heat recovery means include: 1) heat exchangers; 2) heat pumps; 3) compressors; 4) thermal wheel heat recovery systems; 5) heat recuperators; 6) heat pipe technologies; and 7) boiler flue economizers.

Heat exchangers include: 1) tubular (shell and tube); 2) plate exchangers; 3) extended surface exchangers; 4) heat pipe exchangers; 5) regenerative exchangers (fixed matrix, fixed bed, fluidized bed, rotary, recuperative); 6) indirect contact exchangers; 7) direct contact exchangers; 8) parallel flow exchangers; 9) counter-flow exchangers; 10) cross-flow exchangers; 11) single pass exchangers; 12) multi-pass exchangers; 13) gas-liquid exchangers; 14) liquid-liquid exchangers; 15) gas-gas exchangers; 16) condensers; 17) evaporators; 18) micro-heat exchangers; 19) printed circuit exchangers; 20) perforated plate exchangers; 21) scraped surface exchangers; 22) graphite exchangers; 23) Compact exchangers; 24) tube-fin exchangers; 25) continuous fin on tube array exchangers; 26) plate-fin exchangers; 27) brazed plate fin exchangers; 28) air-cooled exchangers; 29) water-cooled exchangers; 30) organics cooled exchangers; and 31) baffled exchangers.

Types of organisms include: 1) Bacteria; 2) Archaea; 3) Protozoa; 4) Algae; 5) Fungi; 6) Viruses; 7) Multicellular animal parasites; 8) Plant cells; and 9) Animal cells. Modification of organisms include: 1) selective isolation; 2) selective growth; 3) selective breeding; 4) cross-breeding; 5) mutagenesis; 6) protoplast fusion; 7) introducing polyploidy; 8) transgenesis; 9) genome editing; 10) hybridization; 11) genetic transformation of or by genomic DNA, plasmid DNA, mitochondrial DNA, 12) Clustered Regularly Interspaced Palindromic Repeats (CRISPR) processes; 13) TALENS processes; 14) Zinc-finger nucleases processes; 15) Conjugation; 16) epigenetic processing by methylation and/or acetylation; 17) post-transcriptional processing; 18) post-translational processing; 19) alterations in carbon partitioning; 20) alterations in transport signal tags for proteins, carbohydrates, and lipids; 21) improved promoters; 22) improved enhancers; 23) improved transcription factors; 24) RNA editing.

Products and/or services that can be rendered using recovered heat include: 1) local heating; 2) local cooling; 3) desalination; 4) aquaculture; 5) providing activation energy for exothermic chemical reactions; 6) providing the energy to drive endothermic chemical reactions; 7) optimizing enzymatic activity; 8) optimizing catalytic activity; 8) sterilizing substances; 9) electricity co-generation; 10) initiating and maintaining the molten state of substances; 11) distillation; 12) enhancing the malleability of substances; 13) tempering and heat treating substances to increase their strength; 14) viscosity reduction; 15) preventing freezing or gelling; 16) seal gas heating; 17) gas phase preservation to prevent condensation; 18) cooking, frying, broiling, baking; 19) kiln treatment of ceramics; 20) drying; 21) wastewater treatment; 22) growing or maintaining micro-organisms that have been modified to produce biomaterials or biofuels; 23) preheating of process air and materials; 24) drinking water source treatment; 25) hydrogen production; 26) degradation of harmful/toxic chemicals or substances; 27) load-following electrical grid energy requirements.

Heat recovery includes: 1) transferring and utilizing heat produced by a combined heat-power (CHP) system; 2) transferring and utilizing heat produced by a system primarily designed to provide power; 3) transferring and utilizing heat produced by an energy co-generation system.

Year-round includes activity: 1) that can potentially be utilized in all four seasons of the year; 2) does not mean that the activity must be continuous for the entire year. There may be interruptions for maintenance, breakdown, repair, refueling, weather, natural disasters, war, civil unrest, hacking, labor actions, regulatory bases, financial reasons, and other reasons.

Substantially cleaning in reference to water includes: 1) reducing or removing partially or completely any contaminants that are harmful to humans, animals, plants, chemical reactions, the environment, machinery, pipes, electrical equipment, plumbing, valves, etc.; 2) filtering; 3) settling by gravity; 3) centrifugation; 4) screening, 5) using permeable or semipermeable membranes; 6) flocculation; 7) precipitation; 8) coagulation; 9) physical and/or chemical destruction; 10) physical and/or chemical reaction; 11) conversion, 12) adsorption; 13) absorption, 14) application of magnetic energy/field; 15) application of electric energy/field; 16) application of heat; 17) distillation; 18) application of light.

Biomaterials include: 1) polymers; 2) bioplastics (e.g. polylactic acids, polyhydroxyalkanoates); 3) fibers; 4) textiles and fabrics; 5) composites; 6) gels; 7) films and coatings; 8) adhesives and sealants; 9) drugs; 10) supplements; 11) biologicals; 12) ceramics and glasses; 13) tissues; 14) cells; 15) enzymes; 16) catalysts; 17) carbohydrates; 18) lipids: 19) proteins; 20) nucleic acids; 21) metals and complexation compounds.

The implementation of the inventive concepts proceeds substantially as follows:
1) Science/technology, business/financing, legal/regulatory analyses.
2) Securing intellectual property and exclusive permits/licenses.
3) Securing financing.
4) Bench chemistry and biology. This is driven largely by the need for approval of the United States Environmental Protection Agency (USEPA) under the Toxic Substance Control Act (TSCA) and the Clean Water Act (CWA).
   a. We have detailed TSCA/USEPA regulatory requirements we must meet,
   b. We have detailed genetic modification protocols to induce organisms to produce valuable biomaterials in sufficient yield to be economically viable,
   c. We have benchmarks to have the algae/organisms adequately remove P/N/C to achieve desired reductions in Lake Erie and other bodies of water.
5) Pilot Plant construction and operation. This applies the lessons learned from the Bench Chemistry and Biology phase and is driven by the need for approval of the USEPA under the TSCA and CWA.
   a. After the USEPA and other regulators give approval for the Bench Chemistry and Biology phase, we proceed to build a Pilot Plant of between 2-10 acres to apply the lessons learned from the Bench.
   b. The goal is to operate the cultivation facility through all four seasons to achieve adequate yields and contaminant results. We have detailed engineering diagrams and work plans for the Pilot Plant and later the large operational facility.
6) Heat Exchanger Installation on the Nuclear Power Plant. From the beginning a Nuclear Team has been working in parallel with the Nuclear Regulatory Commission (NRC) to secure approval to install the heat exchangers on the nuclear power plant under the Atomic Energy Act.
   a. Every nuclear power plant is different is at least a few important ways. Heat exchanger design is an individualized effort. We have detailed protocols for heat exchanger designs and will be working with the NRC for approval to install heater exchangers. Heat exchangers exist on all Pressurized Water Reactors (PWR) between the primary and subsequent loops. We would install our heat exchangers on the final hot leg of the cooling system loops. Nuclear reactors in Pakistan and India have been successfully retrofitted with heat exchangers to introduce desalination capability to nuclear power plants that were not build with that capability initially.
7) Construction of the Algae/Organisms Cultivation and Co-generation Facility. From the beginning the Cultivation Facility Team has been working in parallel with the local land use authorities, departments of natural resources, and other parties to engineer and construct the ponds and facilities to be used for the 1) substantial water cleaning; and 2) production of biomaterials and/or valuable services.
   a. Massive algae cultivation facilities were built in the American Southwest in an effort to produce biofuels. Those facilities were well and successfully engineered but failed economically because cheap fracked natural gas and oil made biofuels price non-competitive. We have detailed engineering diagrams and work plans for the algae/organism cultivation co-generation facilities.
8) Biorefinery. From the beginning, the Biorefinery Team has been working in parallel to engineer, design, and build the biorefinery that will process the biomaterial.
   a. When we have begun producing the bioplastic polymers, we will need a biorefinery to produce advanced polymers with the characteristics necessary for the products into which the bioplastics will be molded or formed. We have detailed work plants for the biorefinery construction or retrofit.

### Additional Products and Services

We also have detailed engineering designs and operating protocols for the production of 1) other biomaterials; 2) PFAS collection and degradation; 3) drug collection and degradation; 4) collection and degradation of other hazardous substances; 5) hydrogen production; 6) industrial degradation and recycling; and 7) the interconnection of nuclear power plants to solar energy installations.

### EXAMPLES

The following examples are merely illustrative and do not in any way limit other applications of the inventive concepts contained herein.

Example 1: Toxic algae blooms in Lake Erie cause severe injury to human health and the environment, yet no effective or economical solutions have been found. The same nitrogen, phosphorous, and carbon (P/N/C) in agricultural run-off that promote the increasing frequency and intensity of such toxic blooms in Lake Erie can serve as nutrients for a production species of algae grown in raceway ponds in an algae cultivation facility located close to a nuclear power plant on the shores of Lake Erie. Building and getting regulatory approval for such an algae cultivation facility is extremely expensive, but the facility could not be utilized to grow the algae in the cold, dark days of late fall, winter, and early spring in the absence of an external source of heat and energy to warm and light the ponds economically. No investors will fund such a costly project if it sits idle for 5-6 months each year.

One or more heat exchangers recover the unused heat from the nuclear power plant and supply it to the algae cultivation facility to allow year-round operation. The algae facility may include an energy co-generation process that provides artificial light when sunlight is absent or not optimal. In the alternative, relatively inexpensive electricity is provided directly by the nuclear power plant.

Prior attempts to grow algae for large-scale production of biofuel and biomass were placed in hot, sunny locations, but were largely unsuccessful because of the high cost of water, energy, and nutrients to feed the algae. In this example, the costs are significantly reduced because the algae cultivation and energy co-generation facility has plentiful free water from Lake Erie. The heat recovered from the nuclear power plant would have otherwise been wastefully vented into the environment and has little on-going cost to the algae cultivation operation. The water pumped from Lake Erie contains the nutrients that feed the algae in the cultivation facility and comes at no cost to the facility. The year-round use of the lake water nitrogen, phosphorus, and carbon by the cultivated algae substantially removes those nutrients from Lake Erie and thereby substantially reduces or eliminates the toxic algae blooms. Carbon dioxide can be added to the algae as a nutrient or substrate, thereby reducing CO2. Animal waste from concentrated animal feeding operations (CAFO's) is a significant source of agricultural run-off and is increasingly the target of regulation. CAFO-related animal waste is also a low-cost source of nutrition for the micro-organisms producing bioplastics or biomaterials.

That cleaning of Lake Erie and reduction of toxic algae blooms is very valuable and is a source of income for the partnership or joint venture between the nuclear power plant and algae cultivation facility. The monetization of the previously unused heat allows the nuclear power plant to remain economically competitive and prevents closure. The free or relatively inexpensive heat, electricity, water, and nutrients make economically viable a valuable algae cultivation facility that would otherwise not exist in northern Ohio and southern Michigan.

Example 2: The interaction between the nuclear power plant and the algae cultivation and energy co-generation facility is much the same as in Example 1, but now with the addition of the production of useful substances by the algae at the cultivation facility. The algae are selectively bred or genetically modified to produce bioplastics or other valuable biomaterials. These serve as a valuable additional source of income for the algae cultivation facility. Toledo and Detroit are blessed with oil refining and petrochemical facilities that could be adapted to the production of bioplastics and/or other biomaterials. The re-engineering of those refineries would be relatively inexpensive compared to new construction. Those petrochemical refineries are also close to the processors and end-users of the bioplastics and biomaterials (e.g. the auto industry). Those oil and petrochemical refineries currently benefit from cheap fracked oil and gas but are under severe economic strain from the ethanol mandates and the current attempt of Michigan to close the Enbridge Oil Pipeline #5 that crosses the Strait of Mackinac. If the price of oil and gas go up or the #5 pipeline is closed, those oil and petrochemical refineries will close unless they can be put to alternative uses (i.e. processing bioplastics produced near the Fermi, Davis-Besse, and/or Perry nuclear power plants).

Example 3: The minimum efficient scale of the combination of nuclear power plants and algae cultivation and energy co-generation facilities are such that it is probable that only 3-5 such facilities would be needed in the United States. The recovery of heat from nuclear power plants could be used to produce other valuable services such as local commercial or industrial uses (e.g. chemical, steel, concrete, metals, refining, energy co-generation, food, oil and gas extraction, manufacturing, or other processes). Only approximately 28% of CO2 production comes from energy generation. The utilization of heat from zero-carbon nuclear power would significantly reduce CO2 generation from other industrial, residential, and commercial processes.

Example 4: An ingenious invention by an MIT professor is a liquid metal battery. The liquid metal battery could solve problems of energy storage for both renewable sources and for nuclear power plants. The problem is that the metals in the battery must be kept in a molten state and the cost of continuously heating those metals makes the liquid metal battery economically infeasible. Heat recovered from nuclear power plants could supply much of the heat needed to make the liquid metal batteries operational and cost effective.

Example 5: Most of the current nuclear power plants were designed and built in the 1960's, 70's, and 80's. As such, they are technically outdated. There are many exciting Generation III and IV nuclear power plants currently being designed and tested. Those new designs will include greater efficiency, safety, and high-grade heat. Breeder and burner versions of those advanced reactors will use current spent fuel (i.e. “nuclear waste”) as their fuel and convert those long-lived isotopes to much shorter-lived radionuclides. In essence, the advanced reactors will provide zero-carbon energy, high-grade heat, and substantially solve the nuclear waste problem. Unfortunately, those advanced designs will not be ready for construction and operation for 10-20 years. By that time many of the current nuclear power plants will have been decommissioned and returned to general land use.

If the Generation III and IV nuclear reactors have to be built on new sites, they may be prohibitively expensive. The simple reality is that the best sites for these extraordinary advanced nuclear reactors are the sites of the currently nuclear reactors. The current nuclear reactor sites have everything the advanced reactors will need: 1) electrical transmission and distribution infrastructure; 2) cooling water as the ultimate heat sink; 3) location far enough away from population centers to minimize the consequences of accidents, but close enough to minimize line loss and transmission costs; 4) human capital in the form of trained and experienced nuclear workforces and expertise; 5) regulatory licenses and permits that will merely need to be amended for the new technology rather than requiring a lengthy and expensive re-vetting at a new site; 6) social and community acceptance. Just about the only places that are receptive to nuclear power plants are the communities in which they already exist. The people appreciate the jobs, taxes, and benefit of nuclear power and it makes it less likely that community resistance and litigation will deter construction; and 7) co-located algae cultivation and energy co-generation facilities or other firms that utilize the heat from the nuclear plants.

After a nuclear power plant is decommissioned, all of the benefits of that site atrophy away. Creating those advantages anew is massively expensive and will be a major impediment to construction of the Generation III and IV nuclear reactors. The algae cultivation and energy co-generation facilities and other uses of unused nuclear heat will keep the current nuclear power plants economically viable long enough for the advanced nuclear reactors to come online and be built into the current sites' advantages.

Example 6: Polyfluoroalkyl and perfluoroalkyl (PFAS) chemicals are widely used as fire retardants. They are called “forever” chemicals because the halogen-carbon bonds are so strong that it is exceedingly difficult to break down those chemicals into their constituent elements. Immense heat and energy are required but are usually very expensive. PFAS chemicals are highly toxic to human beings and are pervasive in the environment. We can chemically separate and collect PFAS chemicals, but the economics of degrading them into harmless constituents is unfavorable. Here we will convert “trash” into treasure. Nuclear waste heat is exactly that, waste. It turns out that excess solar heat and energy at peak midday output are also “waste” in that they exceed the total demand of the electricity grid. Solar energy producers must therefore either 1) harmfully curtail energy off-take from their solar panels (thereby causing long-term heat damage to the semiconductor wafers); or 2) pay electricity consumers (negative pricing) to take their solar electricity output for a few hours each day. That “Duck Curve” hurts the economics of both solar and nuclear energy producers. PFAS chemicals can be separated and stored until very inexpensive or negatively priced solar and nuclear energy are available for several hours each day to economically degrade them. I will either co-locate solar and nuclear power generation with a PFAS degradation facility or transmit excess solar energy to those facilities close to the nuclear power plants. The adjacent bodies of water (or more distant bodies of water if the PFAS chemicals are removed and transported to our PFAS degradation facilities) are cleaned of their PFAS chemicals. This is also true for many non-PFAS chemicals as well.

Example 7: Many pharmaceuticals and their toxic metabolites are excreted into rivers, lakes, groundwater, and other bodies of water. They cause many direct and indirect problems. Anti-retroviral drugs increase the mutational pressure on various viruses that can then become more virulent to all forms of life. Those mutations can drive the emergence of pandemics and insect-borne diseases. Recovered heat and energy from a nuclear power plant alone or in combination with other sources of heat and energy (solar, as in Example 6, above) could be used to break down pharmaceuticals and other contaminants in water.

Example 8: Hydrogen production from electrolysis of water also requires immense amounts of heat and energy. Current Generation I, II, and Ill nuclear power plants do not reach temperatures high enough to economically break water down into hydrogen and oxygen. Using heat compressors to raise the temperatures require too much energy to be commercially practical even with the use of current catalysts. Again, concurrent use of nuclear and solar (or other sources of excess energy, e.g. wind) at their peak production with very inexpensive or negative pricing could make hydrogen production economically viable even for current nuclear power plants.

Example 9: Load following is the ability of an energy generator to rapidly adjust its output to meet the almost instantaneous demands of the grid. Too little electricity and the customers have brownouts or blackouts; too much electricity and the sensitive motors, sensors, transformers, and relays are irreparably damaged. Nuclear power plants are very efficient at producing steady baseload amounts of electricity but have been poor at load following. The same heat exchangers that take off

“waste” heat for algae cultivation farms, PFAS/pharmaceutical/contaminant degradation, and hydrogen production could, when connected to commonly used variable rate generators, rapidly and effectively vary their energy outputs to follow the load requirements of the grid. That in turn allows nuclear power plants to bid for 1) capacity market supply; 2) “next day” short-term supply; and 3) “peaker” very short-term energy supply. The ability to follow the grid's load demands makes nuclear power plants far more competitive with greenhouse gas producing energy sources. The same principles would apply to solar, wind, and other renewable energy sources that currently do not follow grid load requirements well or at all.

I will discuss the technologies, utility, novelty, non-obviousness, enablement, written description, best mode, etc. requirements at greater length in the Information Disclosure Statement (IDS) in the context of the prior art where, with all due respect, those issues are better discussed once rather than twice (as is now the practice). The Information Disclosure Statement is hereby incorporated by reference with respect to all of the inventive concepts, embodiments, and examples contained herein.

What is claimed is:

1) A method comprising
using heat recovered from a nuclear power plant to enhance,
maintaining year-round modified organisms that are,
substantially cleaning water.

2) A method comprising
using heat recovered from a nuclear power plant to enhance,
maintaining year-round modified organisms that are,
producing biomaterials.

3) A method comprising
using heat recovered from a nuclear power plant to enhance,
maintaining year-round modified organisms that are,
producing biomaterials and substantially cleaning water.

4) A method comprising
the methods in claims 1), 2), and 3) in which the heat source is a refinery.

5) A method comprising
the methods in claims 1), 2), and 3) in which the heat source is a combination nuclear power plant and solar energy installation.
